(19) United States
(12) Patent Application Publication
(10) Pub. No.: US 2003/0211043 A1
(43) Pub. Date: Nov. 13, 2003

(54) Eye treatment

(76) Inventor: Donald R. Korb, Boston, MA (US)

Correspondence Address: Robert G. Rosenthal, Law Office of Robert G. Rosenthal, Suite 200, 5856 Faringdon Place, Raleigh, NC, 27609, US

(21) Appl. No.: 10/453,123

(22) Filed: Jun. 3, 2003

Related U.S. Application Data
(63) Continuation-in-part of application No. 09/969,232, filed on Sep. 28, 2001.

(51) Int. Cl. A61K 49/00

(52) U.S. Cl. 424/9.6

(57) ABSTRACT

The invention relates to a method of diagnosing the eye and to methods for subsequent treatment following such diagnosis. The method involves diagnosing a deficiency in the anatomy and performance of the upper eyelid recognizing the impact of this deficiency during blinking on problems such as dry eye, contact lens intolerance and ocular discomfort in general. The invention also involves the use of this diagnostic method to provide a treatment modality to alleviate such problems.

### RELATED APPLICATION

This application is a continuation-in-part of U.S. patent application Ser. No. 09/969,232 filed Sep. 28, 2001

### BACKGROUND OF THE INVENTION

1. Introduction

This invention relates to a method of diagnosing the eye and prescribing subsequent treatment based upon said diagnosis. More particularly, this invention relates to a method for diagnosing a deficiency in the anatomy and performance of the upper eyelid; a recognition of the impact of this deficiency during blinking on problems such as dry eye, contact lens intolerance and ocular discomfort in general; and the use of this diagnostic method to provide a treatment modality to alleviate such problems.

2. Description of the Prior Art

Blinking and the function of the eyelid are of major importance in maintaining the health of the eye.

The eyelids, particularly the anterior surfaces of the lids, protect the eye. The lower lid has a relatively passive role as a consequence of its anatomy and it undergoing limited movement during blinking. This movement consists of a slight upward movement in and towards the nose. For purposes of the discussion that follows, the lower lid is considered essentially stationary and of limited relevance for purposes of the subject invention.

In contrast to the lower lid, during blinking, the upper lid is highly mobile and is responsible for many functions. These functions are dependent upon the ability of the upper lid to move downward, either during a normal blink, or during closure to protect the eye. The role of the upper lid includes protection of the eye by emergency closure; protection of the eye during sleep; and during blinking, the spreading of tears across the ocular surfaces, the wetting of the ocular surfaces, the supplying of oil from the oil glands (meibomian glands), and the spreading of this oil over the surface of the eye, the removal of foreign matter by physical movement, and the polishing and maintenance of the optical surface of the cornea, the latter being a requirement for optimal vision.

It is known that if the cornea is not sufficiently protected by an adequate tear film, the epithelial cells and their tight junctions are compromised, and the cornea and the eye are then subject to a host of complications including infection. Since blinking is crucial to the formation and maintenance of the tear film, blinking is also crucial to comfort, vision and the functioning of the eye. If the upper lid is unable to close shut, the consequences are severe since without blinking or closure of the eye during sleep, the epithelial cells of the cornea and the other exposed surfaces of the eye desiccate resulting in discomfort, tearing, pain and, in severe situations, damage to the epithelial cells and deeper tissue of the cornea, even the possible loss of the eye.

The average blink rate is about 12 blinks per minute. However, it is known to vary depending upon the activities of the individual. This blink rate has been reported in several publications as varying from about 3.5 blinks per minute to as many as about 30 blinks per minute. Ploman; The physiology of the eye and vision. In: Duke-Elder S., ed. System of Ophthalmology, Volume IV. St. Louis, Mo.: Mosby 1968:419; York M, Ong J, Robbins J C. Variation in blink rate associated with contact lens wears and task difficulty. AM J Optom Arch Am Acad Optom 1971;48:461-6; Carney L G, Hill R M; The nature of normal blinking patterns. Acta Ophthalmol (Kbh) 1982;60:427-33; Patel S, Henderson R, Bradley L, Galloway B, Hunter L. Effect of visual display unit on blink rate and tear stability, Optom Vis Sci 1991:68:888-92; Monster A W, Chan H C, O'Connor D. Long-term trends in human eye blink rate. Biotelemetry and Patient Monitg 1978;5:206-22; and Tsubota K, Yamada M, Urayama K. Spectacle side panels and moist inserts for the treatment of dry eye patients, Cornea 1996;13:197-201. Each of the aforesaid publications are incorporated herein by reference for their discussions of blink rate and the description of the results of blinking.

It is an accepted principle that blinking is necessary for eye comfort. For example, in Acosta M C, Gallar J, Belmonte C, The influence of eye solutions on blinking and ocular comfort at rest and during work at video display terminals, Exp Eye Res 1999;68:663-9; it was proposed that “Reduction of eye blink frequency elicited by the performance of a visual task with a computer appears to depend on central neural mechanisms that are quite independent of peripheral sensory inputs”. The authors explain that the decrease in the blink rate increases the activity of the sensory nociceptive terminals on the ocular surface, resulting in eye discomfort. They emphasize that this increased sensory input is strongly inhibited by the neural blinking mechanisms during performance of a computer task, leading to a continuation of the discomfort.

Though it is accepted that blinking is necessary for eye comfort and maintaining the health of the eye, the anatomy of the eyelids and their function during blinking are not fully understood though it has been a subject of interest since ancient times. The anatomy of the eyelid is described in detail in many texts, including a description in The Anatomy of the Eye and Orbit, Eugene Wolff, The Blakiston Company, Philadelphia, 1948:140-94; and in a succinct summary in the text, The Eye in Contact Lens Wear, Second Edition, J R Larke, Butterworth-Heinemann, Oxford, England, 1997:1-4, both incorporated herein by reference for their discussion of the anatomy of the eye.

The anatomy of the eyelid relevant to the subject invention is that portion of the upper lid in contact with ocular surfaces. This portion of the lid may be visualized as a wiping surface roughly analogous to the wiping edge of an automobile windshield wiper blade. This is the portion of the back surface of the upper eyelid that makes direct contact with the ocular surfaces—the cornea and the bulbar conjunctiva. It can only be seen when the upper lid is everted. This area of the lid is covered with squamous epithelial cells. It is believed that there is no accepted anatomical term for this area of the lid and for purposes herein, this area will be subsequently referred to as the “lid wiper” portion of the eyelid.

The literature refers to the portion of the upper eyelid which makes contact with the lower eyelid during blinking or lid closure as the marginal area, starting in the area of the eyelashes and extending backward to the eye where it is noted that a much sharper junction is formed against the surface of the eye, Larke J R. The Eye in Contact Lens Wear, Second Edition, Butterworth-Heinemann, Oxford, England, 1997:2, incorporated herein by reference. However, other authorities utilize the descriptor marginal to also include the area of the lid in contact with the ocular surfaces. Duke-Elder S. System of Ophthalmology, Vol II. Henry Kimptom, London and Kessing SV, incorporated herein by reference. The portion of the upper eyelid that makes contact with the lower eyelid during blinking or lid closure, the marginal area, is illustrated in FIG. 1, 102; FIG. 2, 202; and FIG. 4, 402.

The lid wiper portion of the eyelid cannot be readily observed since it is behind the upper lid and therefore, the physical relationship of this wiping portion of the lid to the eye is simply assumed. The original assumption that the marginal area made contact with the ocular surfaces appears to have originated in the 1904 publication of Parsons J H, The Pathology of the Eye, Vol.I., Hodder and Stoughton, London, 1904, where Parsons assumed that, owing to the squamous type of epithelium in the marginal areas, this part of the eyelid was in particularly close contact with the eye, especially where squamous cells are a feature of anatomical parts of the body that are designed to make contact. It is believed that the physical dimensions and shape of this area are not described in the literature. For example, FIGS. 1 and 2 of the drawings, diagrams from the Wolf text (page 145) and the Larke text (page 2), both cited above, illustrate that the areas of contact with the ocular surfaces are not identified. In FIG. 1, the upper eyelid 100 illustrates the meibomian glands 101. In FIG. 2, the lid 200 is shown, the meibomian glands 201 are shown, but there is no reference to the area where the lid wiper would be found. Other articles relating to the upper lid, blinking, diseased states of the upper lid, and the area of dry eyes, similarly fail to provide detailed information on the nature or physical dimensions of the lid wiper portion of the upper lid.

It is believed that the only investigation of the nature of the contact of the inner aspects of the upper lid with the ocular surfaces was conducted with one subject and published by Kessing S V, A new division of the conjunctiva on the basis of x-ray examination, Acta Ophthalmologica, Copenhagen, 1967;45:680-83. Kessing established that only the so-called marginal area of the upper eyelid was in contact with the eyeball, while for the lower eyelid, the entire inner area was in close contact with the eyeball. A diagram of the upper lid appearing in the Kessing publication is shown as FIG. 3 of the drawings. From the drawing, it can be seen that the area of contact of the upper lid is not specifically identified. A review of Kessing and FIG. 3 shows the lid 300 in contact with the ocular surface 301, but does not reveal physical dimension or other detailed information concerning the lid wiper. All that is reported is the observation from a tomographic section following the application of contrast medium that there was contact of the marginal epithelium of this area of the lid with the eye.

From the above discussion, it can be seen that the knowledge of the lid wiper aspect of the upper eyelid has not significantly progressed since the 1904 assumption by Parsons that it must make contact with the surfaces of the eyeball due to the presence of the squamous epithelium, and the validation of Parson's assumption by Kessing's 1967 study of one subject.

It is known from the literature that the eye is covered with a complex tear film. The tear film protects the cells of the eyeball from drying and damage. As discussed above, blinking is required to cause secretion from the oil glands and to spread the complex tear film over the ocular surfaces to prevent drying. If blinking does not renew the tear film, the cells on the ocular surface, the cornea, and the bulbar conjunctiva, will dry and evidence actual damage. If blinking is voluntarily suspended, within an average of 30 seconds, the eye begins to burn and tear, a protective mechanism to prevent damage.

Practitioners know how to inspect the cells on the surface of the eyeball, and particularly those of the cornea, for compromise and damage resulting from a dry eye condition. The evaluation of the health of the cells of the cornea and ocular surface is usually made with certain staining agents that do not adhere to healthy epithelial cells, but will stain or color compromised cells. After instillation of the two most frequently used staining agents, 2% sodium fluorescein in solution or 1% rose bengal solution, or both, to the tear film, the cells covering the cornea and the ocular surfaces are examined with the magnification of a slit-lamp utilizing filters to intensify the natural fluorescence of these dyes. The damage to the tissue is revealed as “staining”, which is the infiltration of the dye into the cell or between the tight junctions of the cells.

From the above, it is clear that the practitioner knows how to identify and treat the dry eye condition following the onset of the condition. However, this is a remedial treatment procedure. It would be desirable to provide a diagnostic tool capable of identifying the conditions that cause dry eye, preferably prior to the onset of the symptoms of dry eye or at an early stage in the condition.

### DESCRIPTION OF THE DRAWINGS

In the drawings, as described above:

FIG. 1 represents a diagram of the upper eyelid portion in contact with the ocular surface as illustrated by Wolf, supra, with legend removed;

FIG. 2 represents a diagram of the upper eyelid portion in contact with the ocular surface as illustrated by Larke, supra, with legend removed;

FIG. 3 represents a diagram of the upper eyelid portion in contact with the ocular surface as illustrated by Kessing, supra, with legend removed;

FIG. 4 represents a cross sectional diagram of the upper eyelid with the lid wiper shown;

FIG. 5 represents the upper eyelid having been everted with an area of staining illustrating a mild condition of comprise of the lid wiper using a combination of fluorescein and rose bengal where the stained portion is that observable from staining with lissamine green stain;

FIG. 6 is the same as FIG. 5 but illustrating he severe condition where the stained portion is that observable from staining with rose bengal; and

FIGS. 7 through 9 are the same as FIG. 5, but illustrating the results obtained using a combination of stains where FIG. 7 illustrates, as does FIG. 5, that observed by staining with fluorescein, FIG. 8 illustrates that observed by staining with lissamine green, and FIG. 9 illustrates a composite of FIG. 7 superimposed over FIG. 8.

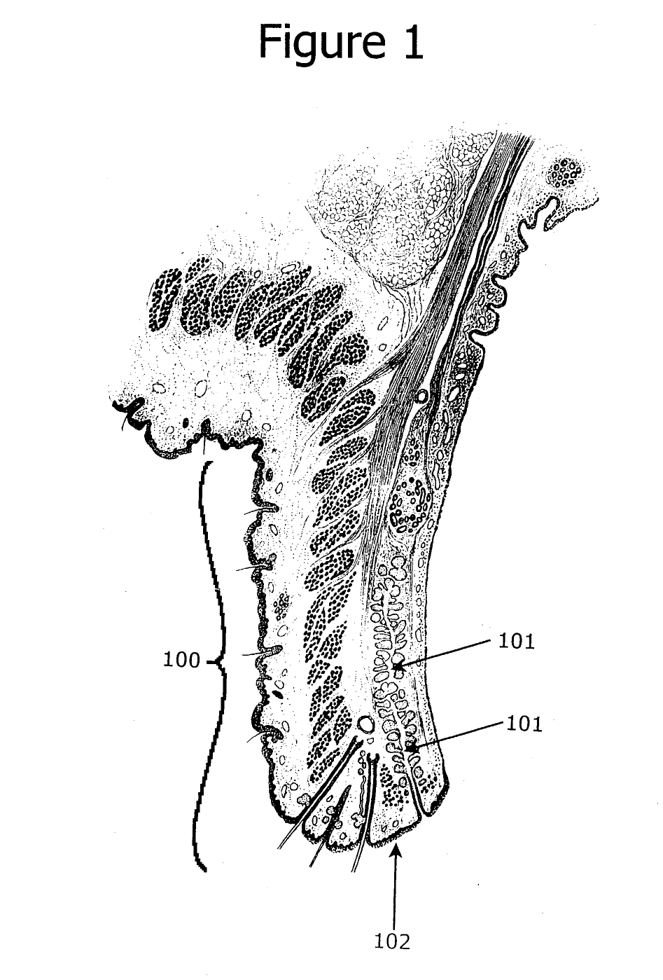

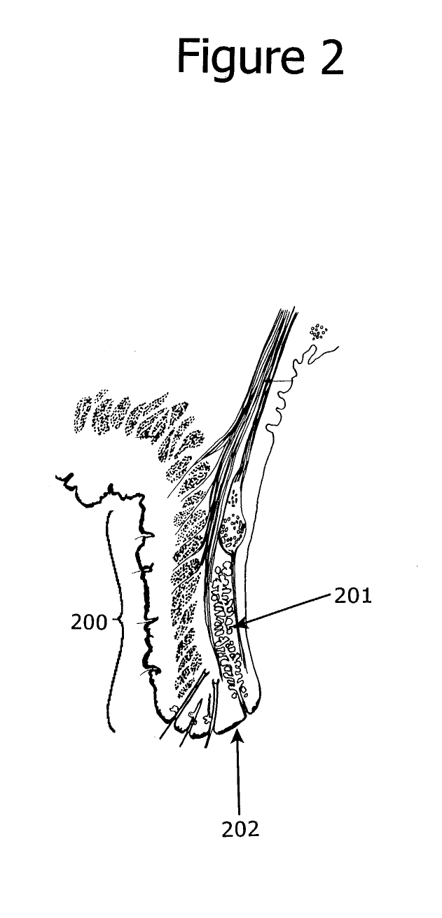

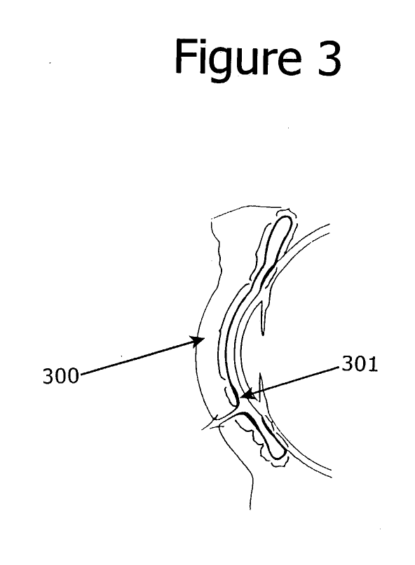

### SUMMARY OF THE INVENTION

The subject invention is based in part upon the discovery that a primary cause of the dry eye state, and the discomfort resulting therefrom, is often a compromise of the cells covering the lid wiper. This compromise may include a broad spectrum of abnormalities such as dead or degenerated epithelial cells to defective epithelial cells.

A further discovery of this invention is that compromised cells on the lid wiper may be readily identified by staining using a conventional stain such as sodium fluorescein, rose bengal, lissamine green, or any one or more stains alone or in combination now known or developed subsequently hereto for such purpose. An additional discovery of the invention is that mixtures of stains are desirably used, each for a specific purpose. Another discovery of the inventions is that diagnosis of compromised cells may be made prior to the actual development of the dry eye state, and prior to the onset of its symptoms. Consequently, the invention provides an early diagnostic tool for the identification of the conditions leading to the dry eye state, and permits the practitioner to initiate an early treatment modality including tear replacement vehicles, lubrication and rewetting agents, wound healing drugs, other treatment modalities for dry eye, and possibly, procedures to immobilize the upper lid to prevent further compromise from the mechanical trauma associated with blinking.

From the above, it can be seen that one object of this invention is to provide a means for identifying or diagnosing compromise of the squamous epithelial surface of that portion of the upper eyelid which makes contact with the ocular surfaces.

A further object of the invention is to provide a means for detecting mild to severe grades of lid wiper epitheliopathy within a broad range of abnormalities.

Another object of this invention is the use of the aforesaid diagnosis to develop a treatment modality for patients suffering compromise of the squamous epithelial surface of the lid wiper.

### DEFINITION OF TERMS

For purposes herein, the term:

“Compromised” epithelial cells means any cell exhibiting an abnormality;

“Defective” epithelial cells means any cell that is damaged, but not dead; and

“Degenerated” epithelial cells means a cell that is dead.

### DESCRIPTION OF THE PREFERRED EMBODIMENTS

Every surface of the body is covered with cells including the lid wiper. The type of cells, are squamous cells as noted by Parsons, supra, in 1904. These cells cover many surface areas of the external body and are designed to make contact and permit rubbing inclusive of the rubbing over the cornea as occurs during blinking.

Blinking involves a great deal of lid movement as the lid passes over the ocular surfaces. If the average blink rate is 12 blinks per minute, there are approximately 11,000 blinks per day, which translates to approximately 4 million blinks per year. The tear film acts as a lubricant for each of these blinks. If the tear film is inadequate as occurs with dry eye states, within a short length of time, the act of blinking, normally without sensation in a healthy eye, evokes sensation and may actually be painful. This is the result of the discomfort or pain associated when the area of the lid wiper is not separated from the ocular surfaces by an adequately thick and appropriate tear film, or by actual physical damage to the squamous cells of the lid wiper from an inadequate tear film and lack of lubrication.

The blinking required to maintain the tear film and the wetting of the corneal surface, in the absence of adequate lubrication, may result in further damage to the squamous cells of the lid wiper. Thus, though blinking may be helpful for the ocular surfaces of the cornea and conjunctiva, it may further compromise the squamous cells of the lid wiper. A patient may not recognize discomfort as occurring with the blink action. Instead, the patient usually describes the discomfort in terms of the classic dry eye symptoms of a scratchy, gritty, sandy, irritative or tired sensation. The patient is subconsciously forced to choose between suspending the blink to prevent this form of discomfort and the resultant discomfort caused by the desiccation of the corneal and ocular surfaces with accompanying sequelae of epithelial compromise and damage. When the condition is acute and severe, burning and tearing occurs as a protective mechanism to provide lubrication to prevent severe damage. Thus, the cause of the discomfort is attributed to dry eyes, or to a specific dry eye state, when the actual cause of the discomfort is physical damage to the squamous cells of the lid wiper.

While the dry eye state causes discomfort, it is a discovery of the invention that a primary mechanism for the discomfort is frequently the condition of the cells covering the lid wiper. These cells become compromised as revealed by staining using conventional stains. The stain may be a single stain or a combination of stains as will be discussed in greater detail below.

The squamous epithelial cells of the lid wiper interface with the tear film of the eye. The surface of the lid wiper (the epithelial cells) must, upon blinking, spread the tear film over the ocular surfaces to achieve wetting and polishing of the ocular surfaces, a very intricate process. The outer surfaces of these epithelial cells, the cell membranes, are protected, as are all epithelial cells related to the surfaces of the eye, by a complex mucoid- like coating that is the actual interface with the tear film.

Dry eye states, regardless of the cause, where the quantity and/or quality of the tear film do not adequately lubricate the epithelial cells of the lid wiper and their protective coatings, result in compromise to the protective coating and the outer membrane of the cells. As the insult exacerbates, as would happen with further dryness and compromise to the tear film, the damage to the cell and the cell-to-cell junctions increase. The outer cell membrane becomes compromised, and damage to the nucleus of the cell can occur. As the damage increases and is present for an extended period of time, individual cell death is likely to occur.

Thus, it is important to maintain an adequate tear film to provide adequate lubrication for the maintenance of the squamous epithelial cells of the lid wiper and their protective coatings.

Ocular stains are used to reveal compromised epithelial cells and/or their associated tight junctions on the ocular surface. The use of stains allows the direct observation of these abnormalities without an extended delay for the processing of laboratory tests. Ocular stains will not penetrate and discolor (stain) healthy viable cells on the ocular surface, but will penetrate and stain compromised epithelial cells and/or their associated tight junctions. Different stains are known to disclose different types of cellular damage. For example, sodium fluorescein dye (fluorescein) has been demonstrated to stain the cell-to-cell junctions and compromised cells of ocular epithelium as well as certain types of mucus secretions. Rose bengal is known to stain degenerated epithelial cells, and to specifically stain the nucleus of the cells though it has a sting potential when applied to the surfaces of the eye sufficiently severe so as to limit its use in routine clinical practice. Lissamine green is considered to have identical staining properties to rose bengal, but without the disadvantages of toxicity and sting potential and therefore, is preferred for the staining of cell nuclei, dead cells and degenerated cells. Thus, different dyes disclose different defects of the cells and establish the magnitude of the damage to the cells and the magnitude of the condition. Therefore, the more severe the staining of any type, the more severe is the particular condition or disorder. If the cell nucleus stains, as will occur with either rose bengal or lissamine green, the damage is more severe than if the only staining is with fluorescein since fluorescein staining indicates that the damage is to the more superficial areas of the cells and/or to the cell-to-cell junctions. Thus, the use of multiple stains to accomplish both purposes has obvious advantages in diagnosis. While dry eye states are universally recognized to affect the vitality of the epithelial cells covering the surfaces of the eyeball, it is believed that prior to the invention described herein, stains have not been used to determine the condition of the epithelial cells of the lid wiper.

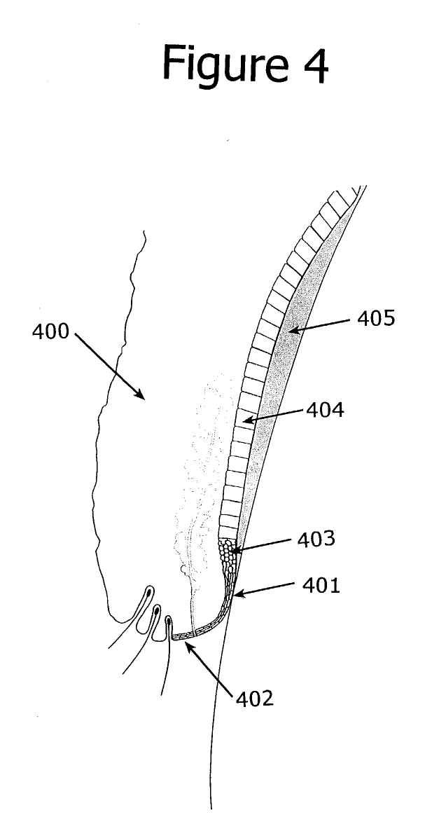

The position of the lid wiper on the upper eyelid and the location of the squamous cells is illustrated in FIG. 4 of the drawing which is a cross sectional diagram of the upper eyelid 400. The lid wiper 401 is the small area that would be in relative contact with the ocular surfaces. In use, it is separated from the ocular surfaces by a boundary layer of tear fluid, not shown. The exact dimensions of the boundary are not known. It is thought that this boundary tear fluid could be as thin as 1μ or as thick as the usual tear film that is reported to be in the range of 5 to 10μ. The marginal conjunctiva 402, and the lid wiper 401, are covered with squamous epithelium, a type of epithelium designed for contact. As the epithelium continues upward on the inner surface of the lid from the area of the lid wiper, it changes from the squamous type of epithelial cell to transitional 403 and then to columnar 404. The area of the upper lid, which has columnar cells, is not in contact with the ocular surfaces, the space between the columnar cells and the ocular surfaces is termed Kessing's space 405.

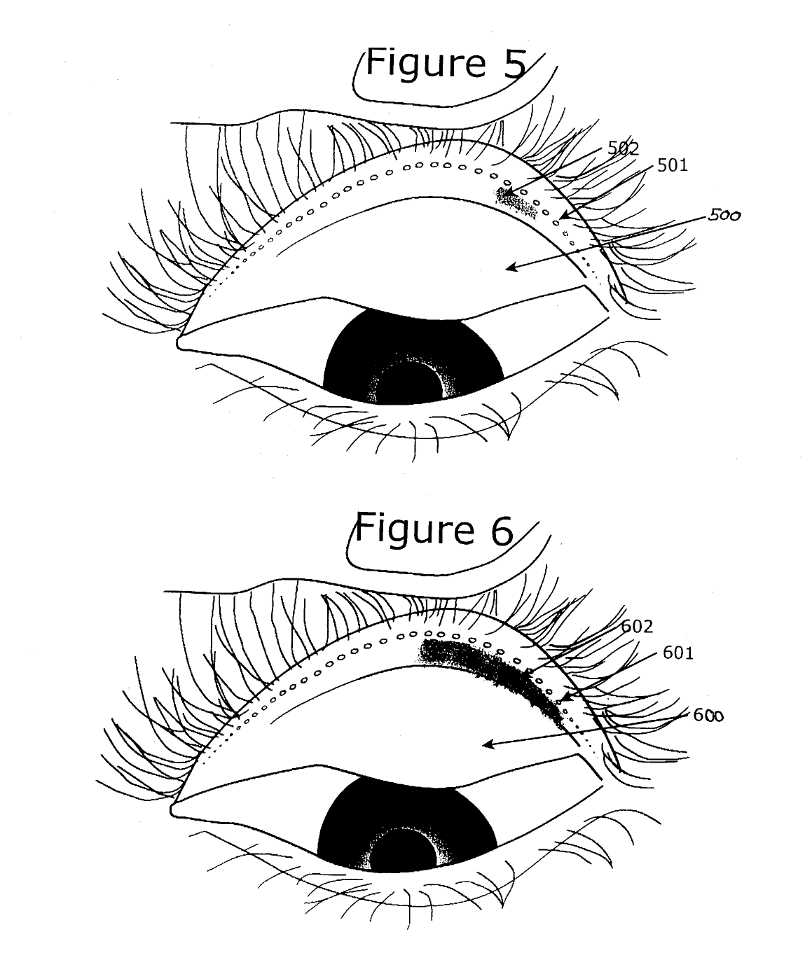

FIGS. 5 and 6 of the drawings diagrammatically represent the upper eyelid 500 and 600, respectively, after having been everted, with the area of staining illustrated for a mild [FIG. 5] and severe [FIG. 6] condition of compromise to the lid wiper. The circular orifices of the Meibomian glands 501 and 601, adjacent to the eyelashes, appear superior to the area of the lid wiper since the lid in each of the conditions is everted. The area of compromise to the squamous epithelium of the lid wiper, 502 and 602, as evidenced by staining of the tissue, is illustrated as areas of different color, with the normal epithelial color being represented as white. The areas of infiltration of the epithelium by the elucidating dyes would appear in color where the color is determined by the dye used. The area would be yellow-green when stained with fluorescein, and red when stained with rose bengal. The smaller area 502 in FIG. 5 represents mild compromise while the larger area 602 in FIG. 6 represents a more severe condition.

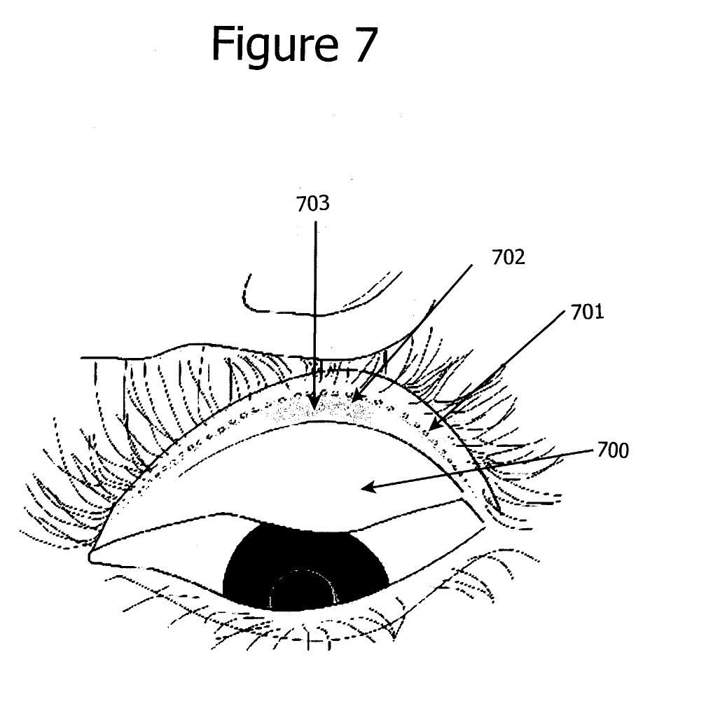

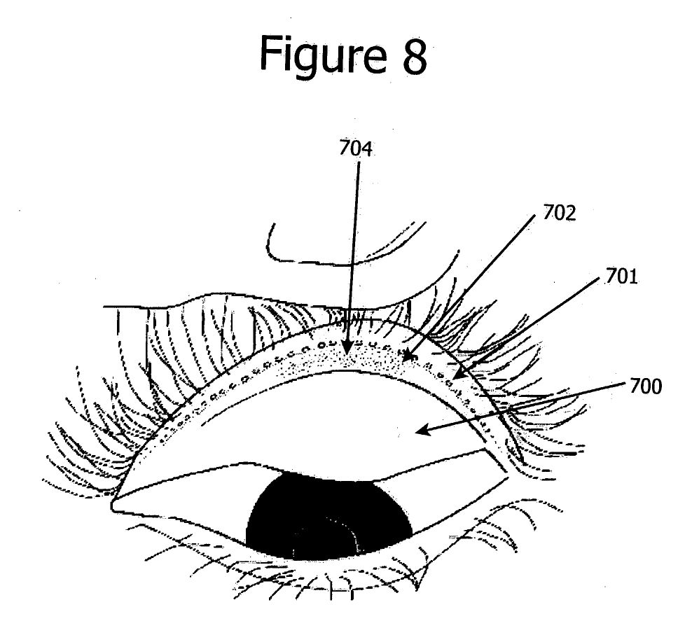

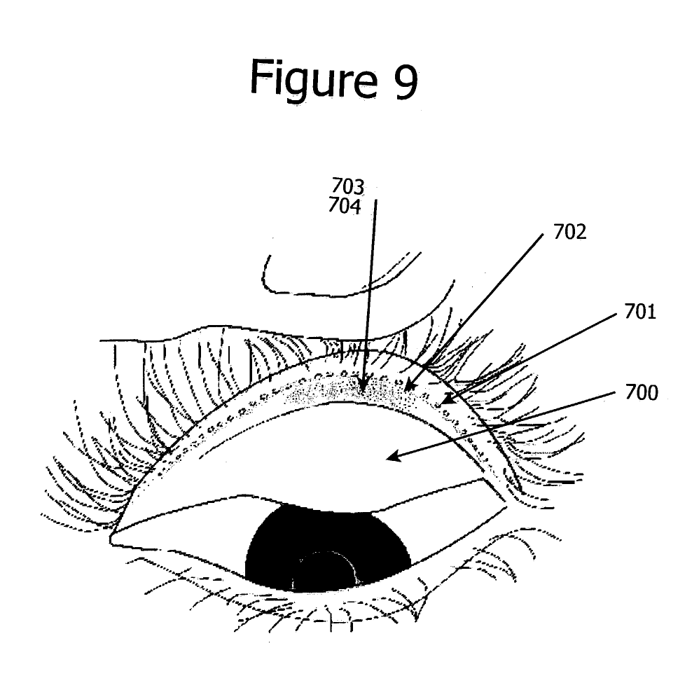

FIGS. 7 through 9 of the drawings diagrammatically represent a composite of the upper eyelid 700 everted after the instillation of a combination of stains into the eye. In this instance, the combination of stains was fluorescein and lissamine green. As above, the circular orifices of the Meibomian glands 701, adjacent to the eyelashes, appear superior to the area of the lid wiper. The area of compromise to the squamous epithelium of the lid wiper 702, as evidenced by the combined action of the stains on the tissue, is illustrated as differing areas of color, with the normal epithelial color being represented as white. The areas of infiltration of the epithelium by the elucidating stains would appear in color where each color is determined by the particular stain or the combination of stains used. As represented, there is a yellow-green area 703 stained with fluorescein [FIGS. 7 and 9] appearing as a diffuse stained surface over the squamous epithelium, and as a punctate area of green spots 704 [FIGS. 8 and 9] within the yellow green diffuse area stained with lissamine green. It should be understood that a practitioner would not observe the squamous epithelium as it is represented in FIG. 9 when doing an examination. Fluorescein is viewed under black light while the lissamine green is viewed under illuminated white light so that the two areas would have to be viewed separately. To obtain a representation as shown by FIG. 9, the practitioner would have to record an image of the fluorescein stained area, a separate image of the lissamine green image, and overlay one on the other to obtain the composite of FIG. 9 using art recognized procedures.

FIG. 9 illustrates differential cell damage within the same squamous epithelium area of the lid wiper. The diffuse yellow green area stained with the fluorescein dye comprises damaged or abnormal cells while the green punctate dots represent severely damaged or dead cells. The reason for the difference is the way in which the stains interact with the cell. Lissamine green is believed to penetrate into the nucleus of the cell while fluorescein is believed to react with the cell wall or the coating over the cell wall.

The cells of the lid wiper may become compromised although the eye does not suffer from a dry eye condition. For instance, an individual may have an adequate tear film and not exhibit dryness, unless engaged in computer activities. The computer use may result in compromise to the lid wiper because of the reduced blink rate and temporarily limited lubrication to the lid wiper. In such cases, the cells may recover in as little as 1 to 2 hours, although most frequently recovery requires 3 to 12 hours. In certain instances, a single session of intense computer use may require up to 2 weeks to recover. Since the approximate 10,000 blinks per day tend to inhibit healing because of the physical motion of the lid wiper on the surfaces of the eye, the result is that it is possible to engage in only about 1 or 2 relatively limited computer sessions per week, or other analogous activities, to cause a compromise of the lid wiper and the discomfort resulting therefrom.

It is believed that examination of the cells of the lid wiper has never been advocated nor is it obvious to examine these cells. This area is not visible with the usual examination techniques. The examination of the outer cells of the cornea, the epithelial cells, is readily achieved in clinical practice by instilling dyes into the tear film, since these cells are exposed when the eyes are open. After 10 to 60 seconds following instillation of the dye, the cells are examined with the slit-lamp microscope, utilizing colored filters to enhance the fluorescence. Areas of compromised cells are immediately visible, since the dye infiltrates the compromised cells and is seen as areas of fluorescence, a phenomenon that does not occur with healthy cells. These procedures are readily mastered and are a part of routine clinical practice.

The lid wiper is not visible without the physical eversion of the upper lid because it is located on the back surface of the upper lid unlike the external surfaces of the exposed eyeball which are exposed and readily visible when the eyes are open. See FIG. 4. Therefore, examination of the lid wiper requires eversion of the upper lid to bring the area of the lid wiper into view. However, examination of the area of the lid wiper with cellular damage after eversion of the upper lid is not revealing when examined with the magnification of the slit-lamp microscope unless elucidating dyes or stains are used. In other words, it is necessary to achieve staining of the cells of the lid wiper with one or more diagnostic staining dyes to observe the phenomenon and to make the diagnosis of lid wiper staining (disease).

The method used to stain the lid wiper is relatively simple. The concept is similar to that used for the staining of the ocular surfaces. The first step is to apply dye to the tear film prior to eversion of the upper lid. The dye may be a single dye or a mixture of dyes. It is necessary to allow the usual blinking processes to distribute the elucidating dye or dyes throughout the tear film and to rub the tear film with the dissolved dye against the lid wiper. If the squamous epithelium of the lid wiper is not compromised, there will be no visible staining, however, if the epithelium is compromised the stain will infiltrate the tissue and the stained tissue will be visible after the lid has been everted and the lid wiper examined with the slit-lamp and filtered examination light.

In the staining procedure, a minimum dose is applied to the tear film prior to eversion of the upper lid. For dyes conventionally used in this procedure, especially fluorescein, this dose may vary between about 1 and 100 μl of a 0.5 to 5 percent by weight of the solution and preferably varies between about 5 and 50 μl of a 1 to 3 percent by weight of the solution. However, the dose will vary with the specific dye that is utilized and the condition of the eye, greater compromise requiring lesser dose. Smaller doses of rose bengal are desirable, usually between 2 and 20 μl of a 0.5 to 3 percent by weight solution, since the rose bengal may produce dose related stinging. Lissamine green may be used in amounts equivalent to fluorescein, as it does not induce stinging when applied to the eye. When a combination of dyes is used in a single combined treatment, it is desirable that the dose remains within the limits of 1 to 100 μl.

With all dyes currently used for this purpose, the minimum dose would be at least 2 μl. Further, one application or a minimal dose of the stain may not infiltrate the cellular defects in the lid wiper tissue, since the blinking action may remove the stain from the tear film and may not allow adequate contact time for the stain to infiltrate the cells. For this reason, it may be necessary to use a technique of two to three sequential applications of a dye prior to the eversion of the upper lid to allow adequate contact time for the stain to infiltrate the tissue of the lid wiper whereby it can be detected. The sequential applications of the stain should be at 3 to 5 minute intervals to maintain a high concentration of the elucidating dye in the tear film where it can be presented to the lid wiper with each blinking action. Thus, the examination of the lid wiper requires a specific technique for detection of lid wiper disease.

In the preferred embodiment of this invention, the dye comprises a combination of fluorescein with either rose bengal or lissamine green. In the most preferred embodiment of the invention, the combination comprises fluorescein and lissamine green. This combination is preferred to the combination of fluorescein with rose bengal, as rose bengal has been found to sting when applied to the eye in suitable dosage. Lissamine green does not significantly sting in suitable dosage. By elimination of stinging through the use of the lissamine green, a larger total concentration of dye may be added to the eye and therefore, the fluorescein and lissamine green can be combined and used in a single application for treatment.

It should be understood that the dyes identified for use in practice of the subject invention are dyes conventionally used. However, it should also be understood that as new dyes are developed or approved for ocular examinations, these new dyes may be suitable for substitution for the dyes identified herein for lid wiper examination. Thus the invention should be viewed as a method of diagnosis involving lid wiper examination with one or more suitable dyes that stain damaged epithelial cells.

Three studies were performed to illustrate the above discussion—i.e., to evaluate whether ocular discomfort was associated with the condition of the epithelial cells of the lid wiper, the area of the upper lid that makes contact with the ocular surfaces. These studies are discussed below.

Study 1: This study compared the condition of the lid wiper of patients reporting dry eye symptoms (scratchy, sandy, gritty eyes and/or burning and tearing) to the condition of the lid wiper for patients without any symptoms of discomfort. Contact lens wearers were not permitted in this study.

### Study 1: Study of Patients with Dry Eye Symptoms Compared to Patients Without Dry Eye Symptoms

Methods

Consecutive patients presenting for examination were classified into two groups. The primary criterion for admission to the first group was the presence of one or more of the 5 classical dry eye symptoms of scratchy, sandy, or gritty eyes or burning or tearing. Patients with the diagnosis of Sjogren's disease, rheumatoid arthritis, or other systemic conditions associated with dry eye symptoms were excluded from the study. The two groups were matched for age and sex. The symptoms were qualified into three grades, slight, moderate, and severe. One point was awarded for each grade of severity for each of the five symptoms, resulting in a possible score of 1 to 15. A minimum score of 5 points was required for admission to the study. Patients with scores of 3 or 4 were not admitted into the study.

Clinical Procedure One 40 μl drop of 2% unpreserved sodium fluorescein solution was instilled into the inferior formix.

Following a wait of 3 minutes, a second 40 μl drop was instilled.

Two minutes following the instillation of the second drop the upper lid was everted.

The examination of the area of the lid wiper was then immediately conducted with a Haag-Streit 900 slit-lamp using a cobalt filter and 16 magnification.

A grading scale of no staining to grade 3 staining was used. This classification was made by evaluating the linear area of involvement of the staining according to the following criteria:

| Linear Area of Involvement | Grade |
|---|---|
| less than 1 mm | 0 |
| 1-3 mm | 1 |
| 4-8 mm | 2 |
| over 9 mm | 3 |

The severity of the staining was graded utilizing the normal clinical routine for severity of staining of the corneal epithelial cells as follows:

| Severity of Staining | Grade |
|---|---|
| absent | 0 |
| mild | 1 |
| moderate | 2 |
| severe | 3 |

A final grade was the average of the individual grades for the linear area and the involvement or severity of staining.

At the conclusion of the latter examination, the lid was returned to its normal position and 5 μl of unpreserved 1% rose bengal solution was instilled into the inferior fornix. The examination was repeated using white and red free light. Scoring was as previously described. The scores for the fluorescein and rose bengal examinations were then averaged for the final score.

Results

Thirty patients with symptoms and thirty patients without symptoms were studied. The results are presented in tabular form.

| Average Grade | Distribution of | Distribution of |
|---|---|---|
| of Staining for | Symptomatic Subjects | Asymptomatic Patients |
| Fluorescein and | as a % of Symptomatic | as a % of Asymptomatic |
| Rose Bengal | Population | Population |
| No Staining | 20% | 93% |
| 0.25 to 1.0 | 33% | 7% |
| 1.25 to 2.0 | 27% | 0% |
| 2.25 to 3.0 | 20% | 0% |

There was an obvious difference both in the prevalence and the severity of staining of the lid wiper for patients with symptoms than for patients without symptoms. Of critical importance is that approximately 50% of all symptomatic patients demonstrated moderate grade 2 or severe grade 3 staining, as compared to 0% for those without symptoms. These results proved to be highly statistically significant.

The width of the lid wiper extends for the full width of the entire upper lid. However, the width (height) of the lid wiper in contact with the ocular surfaces in not known. The width of the area of the lid wiper, which stained in these studies, varied from 0.25 mm to 1.5 mm. The linear area of involvement varied from <1.0 mm to >15.0 mm. It should be noted that staining of the lid wiper has been differentiated from a normal staining phenomenon termed Marx' line. The line of Marx runs the entire length of the lid margin of the upper lid just behind the orifices of the meibomian glands. This line stains most acutely with rose bengal, however, it may also stain with fluorescein. It is easily differentiated from staining of the lid wiper, since it is located a significant distance anterior to the area of contact with the upper lid.

Study 2: This study investigated whether ocular discomfort occurring with contact lens wearing was associated with the condition of the epithelial cells of the lid wiper, by comparing the condition of the lid wiper of contact lens wearers with symptoms to the lid wiper of contact lens wearers without symptoms.

### Study 2: Study of Contact Lens Wearers With Symptoms Compared to Contact Lens Wearers Without Dry Eye Symptoms

Methods

Consecutive soft contact lens wearers presenting for examination were classified into two groups. The primary criterion for admission to the first group (asymptomatic group) was a reported daily wearing time of 12 or more hours without symptoms. The primary criterion for admission to the second group (the symptomatic group) was a presence of symptoms that occurred within the first four hours of the wearing of their “best fit” contact lenses. The systoms were classified in the four grades as follows:

| Grade | Comfort Description |
|---|---|
| 1 | Eyes comfortable - feels like you have a pair of comfortable |
| | shoes on, if told to remove when getting home you would forget |
| | half the time |
| 2 | Aware of eyes - like having a pair of dress shoes on, are |
| | tolerable but you would take them off as soon as you got home |
| 3 | Eyes uncomfortable - you would only wear the shoes to an |
| | important party |
| 4 | Eyes intolerable - you would wear the shoes only to |
| | “the ceremony” |

Patients with grades 2, 3 or 4 were accepted into the study. Patients with grade 1 were not admitted into the study.

All patients were examined following the wearing of the contact lenses on the day of the examination for a minimum of 5 hours. At the time of the examination the contact lenses were removed. The clinical procedure was as follows:

One 40 μl drop of 2% unpreserved sodium fluorescein solution was instilled into the inferior fornix.

Following a wait of 3 minutes, a second 40 μl drop was instilled.

Two minutes following the instillation of the second drop the upper lid was everted.

The examination of the area of the lid wiper was then immediately conducted with a Haag-Streit 900 slit-lamp using a cobalt filter and 16 magnification.

A grading scale of no staining to grade 3 staining was used. This classification was made by evaluating the linear area of involvement of the staining according to the following criteria:

| Linear Area of Involvement | Grade |
|---|---|
| less than 1 mm | 0 |
| 1-3 mm | 1 |
| 4-8 mm | 2 |
| over 9 mm | 3 |

The severity of the staining was graded utilizing the normal clinical routine for severity of staining of the corneal epithelial cells as follows:

| Severity of Staining | Grade |
|---|---|
| absent | 0 |
| mild | 1 |
| moderate | 2 |
| severe | 3 |

A final grade was the average of the individual grades for the linear area and the involvement or severity of staining.

At the conclusion of the latter examination, the lid was returned to its normal position and 5 μl of unpreserved 1% rose bengal solution was instilled into the inferior fornix. The examination was repeated using white and red free light. Scoring was as previously described. The scores for the fluorescein and rose bengal examinations were then averaged for the final score.

Results

Twenty-five contact lens wearers with symptoms of discomfort and intolerance meeting the criteria for the study and 25 contact lens wearers without symptoms were studied. The results follow.

| Average Grade | Distribution of | Distribution of |
|---|---|---|
| of Staining for | Symptomatic Contact | Asymptomatic Contact Lens |
| Fluorescein and | Lens Wearers as a % of | Wearers as a % of |
| Rose Bengal | Symptomatic Population | Asymptomatic Population |
| No staining | 16% | 88% |
| 0.25 to 1.0 | 24% | 8% |
| 1.25 to 2.0 | 36% | 4% |
| 2.25 to 3.0 | 24% | 0% |

There was an obvious difference both in the prevalence and the severity of staining of the lid wiper epithelial cells for contact lens wearers with symptoms than for contact lens wearers without symptoms. Of paramount importance is that 60 percent of the symptomatic contact lens wearers demonstrated moderate grade 2 or severe grade 3 staining of the lid wiper, as compared to only 4 percent of the asymptomatic contact lens wearers. These results proved to be highly statistically significant.

### Study 3: Study of Patients with Dry Eye Symptoms Compared to Patients Without Dry Eye Symptoms Utilizing a Mixture of fluorescein and lissamine green dye solution

This study is a repeat of study 1 using a mixture of fluorescein and lissamine green dye solution in place of separate instillations of fluorescein and rose bengal.

Methods

Patients were selected following the procedures of Study 1 with patients having a score ≦2 points entered into the asymptomatic group. Patients with scores of 3 or 4 were not admitted into the study.

Clinical Procedure

One drop, approximately 10 to 20 μl of an unpreserved mixture of 2% sodium fluorescein and 1% lissamine green solution was instilled into the inferior fornix of subjects classified into symptomatic and asymptomatic groups by subjective symptoms.

Following a wait of 3 minutes, a second drop between 10 and 40 μl was instilled. The upper lid was everted two minutes after the instillation of the second drop in order to examine the area of the lid wiper.

The examination for fluorescein staining of the area of the lid wiper was then conducted 60 seconds after the second drop with a Haag-Streit 900 slit-lamp, utilizing a cobalt filter over the light source and 16 × magnification. The examination for lissamine green staining of the lid was then conducted with white light; the change from the cobalt filter to the white light source requires only 1 to 3 seconds to turn the control knob to remove the filter from the path of the illumination of the slit-lamp.

A grading scale of no staining to grade 3 staining was used. This classification was made by evaluation of the linear area of involvement of the staining according to the following criteria:

| Linear Area of Involvement | Grade |
|---|---|
| less than 1 mm | 0 |
| 1-3 mm | 1 |
| 4-8 mm | 2 |
| over 9 mm | 3 |

The severity of the staining was graded utilizing the normal clinical routine for severity of staining of the corneal epithelial cells as follows:

| Severity of Staining | Grade |
|---|---|
| Absent | 0 |
| Mild | 1 |
| Moderate | 2 |
| Severe | 3 |

A final grade was the average of the individual grades for the linear area and the involvement or severity of staining.

Results

Fifty (50) subjects with symptoms and fifty (50) subjects without symptoms were studied. The results are presented in tabular form.

| Average Grade | | |
|---|---|---|
| of Staining for | Distribution of | Distribution of |
| Fluorescein and | Symptomatic Subjects | Asymptomatic Subjects |
| Lissamine | as a % of in Symptomatic | in as a % in Asymptomatic |
| Green Mixture | Population (n = 50) | Population (n = 50) |
| No Staining | 8% | 82% |
| Grade 0.25 to | 28% | 14% |
| 1.0 | | |
| Grade 1.25 to | 44% | 4% |
| 2.0 | | |
| Grade 2.25 to | 20% | 0% |
| 3.0 | | |

There was an obvious difference both in the prevalence and the severity of staining of the lid wiper for patients with symptoms than for patients without symptoms. Of particular relevance is that 64% of all symptomatic patients demonstrated moderate grade 2 or severe grade 3 staining, as compared to 4% for those without symptoms. These results proved to be highly statistically significant.

The lid wiper extends across the full length of the entire upper lid, approximately 25 to 35 mm. However, the precise height of the linear area of the lid wiper, that area in vertical contact with the ocular surfaces is not known. The height of the area of the lid wiper, which stained in these studies, varied from 0.25 mm to 1.5 mm. The length of the area of involvement varied from <1.0 mm to >20.0 mm. It should be noted that staining of the lid wiper can be readily differentiated from a normal staining phenomenon termed Marx'line. The line of Marx runs the entire length of the lid margin of the upper lid just behind the orifices of the meibomian glands. The line of Marx stains most acutely with rose bengal, it stains similarly with lissamine green, and to a lesser degree may also stain with fluorescein. It is easily differentiated from staining of the lid wiper, since it is located a significant distance anterior to the area of contact with the upper lid and may be observed without eversion of the upper lid.

In summary, there was an obvious and statistically greater difference, both in the prevalence and the severity of staining of the lid wiper, for patients with symptoms than for patients without symptoms. This result validates the clinical use of a mixture of fluorescein and lissamine green solution for the diagnosis of lid wiper epitheliopathy, and thus, for revealing the presence of an underlying dry eye condition.

In the above studies, the dye was added in sequential instillations as this provided greater accuracy for clinical testing. However, for diagnostic purposes, in the interest of time and patient comfort, one instillation of a dye, either a single dye or a mixture of dyes, may be preferred.

The above studies demonstrate that dry eye symptoms are highly correlated to compromise and staining of the epithelial cells of the lid wiper of the upper lid. Similarly, for contact lens wearers, ocular discomfort and contact lens intolerance occurring after only four hours of wearing are highly correlated to compromise and staining of the epithelial cells of the lid wiper. These symptoms, which are confused with symptoms of dry eye, from the ocular surfaces, are the result of compromise to the lid wiper, and despite the symptoms, all tests for dry eye may be totally normal. This is the result of the compromise to the lid wiper being caused by an exacerbating condition, such as computer or analogous activities resulting in a temporarily deficient tear film and lubrication of the lid wiper, although the basic tear film status is normal and adequate for almost all normal tasks and circumstances. An examination of the lid wiper is therefore a necessary part of any ocular contact lens examination when discomfort is present.

The discovery of readily identifiable compromise and/or disease processes to the lid wiper permits the diagnosis, treatment, and research of this malady and its causes. For instance, the diagnosis of lid wiper staining and/or disease presents a method to determine whether contact lens fittings or ocular surgical procedures, such as corneal refractive surgery (i.e., LASIK) should be considered. Significant lid wiper compromise presents a contra-indication to contemporary LASIK surgery, and also suggests a lower probability of successful contact lens fitting. Appropriate treatment is required for these situations. Lid wiper compromise also indicates specific treatment modalities, including tear replacement vehicles, lubricating and rewetting agents, wound healing drugs, other treatment modalities for dry eye, and possibly procedures to immobilize the upper lid to prevent further compromise from the mechanical trauma associated with blinking.

In addition to the above, the subject invention may be useful for obtaining regulatory approval for new drug applications. For example, the Food and Drug Administration (FDA) requires clinical studies for the approval of any treatment substance to be marketed with claims of efficacy for a particular problem. With respect to ocular treatment formulations, these studies must prove that there is a statistically significant improvement for one symptom and for one objective sign (ocular finding) if the sponsoring company desires to market the product with claims. This is true regardless of how simple the treatment modality may be. For example, if a saline solution were the new treatment modality and the sponsoring company desired claims, it would be necessary to conduct clinical studies to prove the statistical improvement of one symptom and one objective sign.

The FDA considers any new substance or combination of existing substances a new drug for the approval process. The problem in the past has never been with obtaining improvement for the symptom, but in obtaining improvement for the required one objective sign. The difficulty stems from the fact that all objective tests for the required objective sign are not adequately sensitive enough to show any differences with treatment, despite the fact the product may alleviate, and frequently totally alleviate, all of the patient's symptoms related to the dry eye condition. As a result, companies may be forced to study multiple patients over many years to achieve the required statistical significance for the objective sign necessary to obtain FDA approval for a dry eye treatment drug. The cost of such a study is typically in millions in addition to the lost time for both opportunity and patent life.

The reason for the above difficulties is that current objective tests do not adequately correlate to symptoms and it is difficult to prove that any objective finding is improved by a new modality seeking FDA approval. With lid wiper methodology as described herein, it is possible that there will be a quantitative sign available for all dry eye conditions thus permitting FDA approval with fewer patients over a shorter time period as the methodology will provide a clear end point that is correlated to the status of the tear film and to patient symptomatology.

1. A method for diagnosing the health of the eye, said method comprising the steps of staining the tear film with a staining dye, everting the upper eyelid, and observing the lid wiper portion of the everted eyelid for infiltration of the staining dye into the cells thereof.

2. The method of claim 1 including the step of prescribing a treatment modality for an eye found to have lid wiper compromised or degenerated cells.

3. The method of claim 2 where the treatment modality is selected from the group including use of eye treatment agents or procedures to immobilize the upper lid.

4. The method of claim 2 where the treatment modality includes periodic application of a tear replacement vehicle to the corneal surface.

5. The method of claim 2 where the treatment modality includes periodic application of a tear lubrication or rewetting agent to the corneal surface.

6. The method of claim 2 where the treatment modality is dry eye treatment.

7. The method of claim 2 where the treatment modality includes fitting a patient with a contact lens.

8. The method of claim 1 used for end point determinations for clinical trials.

9. The method of claim 1 where a single dye is applied to the eye.

10. The method of claim 1 where a combination of dyes is applied to the eye.

11. The method of claim 10 where the combination of dyes comprises a dye intended to infiltrate defective cells and a dye intended to infiltrate degenerated cells.

12. The method of claim 11 where the combination of dyes is added as a single dose.

13. The method of claim 11 where the combination of dyes is added in sequential applications where there is a preselected time interval between applications.

14. The method of claim 11 where the staining dye is selected from the group consisting essentially of a dilute solution of sodium fluorescein, rose bengal, lissamine green and mixtures thereof.

15. The method of claim 11 where the dye intended to infiltrate defective cells is fluorescein.

16. The method of claim 15 where the dye is a mixture of fluorescein and lissamine green.

17. The method of claim 1 where the dye is used in an amount of at least 1 μl per application of dye.

18. The method of claim 17 where the dye solution is added in a dose of from 1 to 100 μl.

19. The method of claim 18 where the dye solution is added in a dose of from 5 to 50 μl.

20. The method of claim 17 where the concentration of the dye in the dye solution varies from 0.5 to 5 percent by weight.

21. A composition for diagnosing the health of the eye, said composition comprising a solution containing a combination of dyes where one of said dyes is a pharmaceutically acceptable dye capable of staining lid wiper defective epithelium cells and another of said dyes is a pharmaceutically acceptable dye capable of staining lid wiper degenerated epithelium cells.

22. The composition of claim 21 where the staining dye is a mixture of dyes selected from the group consisting essentially of sodium fluorescein rose bengal, and lissamine green.

23. The composition of claim 21 where the dyes comprise a mixture of fluorescein and lissamine green.

24. The composition of claim 21 where the dose of the dye solution is in an amount of at least 1 μl.

25. The composition of claim 24 where the dose is from 1 to 100 μl.

26. The composition of claim 25 where the dose is from 5 to 50 μl.

27. The composition of claim 21 where the concentration of each dye in the dye solution varies from 0.5 to 5 percent by weight.

28. A pharmaceutical package, said package containing a combination of dyes where one of said dyes is a pharmaceutically acceptable dye capable of staining lid wiper defective epithelium cells and another of said dyes is a pharmaceutically acceptable dye capable of staining lid wiper degenerated epithelium cells.

29. The package of claim 28 where the dyes within the package are selected from the group consisting essentially of sodium fluorescein, rose bengal, and lissamine green.

30. The package of claim 28 where the dyes comprise fluorescein and lissamine green.

31. The package of claim 30 where the dyes are mixed as a single solution.

32. The package of claim 30 where the dyes are separate solutions.

33. The package of claim 28 where the dose of a dye solution is in an amount of at least 1 μl.

34. The package claim 33 where the dose is from 5 to 50 μl.

35. The composition of claim 34 where the concentration of each dye in solution varies from 0.5 to 5 percent by weight.
